SEQUENCE LISTING (5 sequences)

SEQ ID NO: 1 - DNA, 98 bp, synthetic construct
  source 1..98: mol_type = other DNA
ctttacttactacacctcagatatatttcttcatgaagacctcacagtaaaaataggtgattttggtctagctacagaagaaatctcgatggagtggg

SEQ ID NO: 2 - DNA, 98 bp, synthetic construct
  source 1..98: mol_type = other DNA
ctttacttactacacctcagatatatttcttcatgaagacctcacagtaaaaataggtgattttggtctagctacagatgaaatctcgatggagtggg

SEQ ID NO: 3 - DNA, 28 bp, synthetic construct
  source 1..28: mol_type = other DNA
cagctagagctccagtatcgagatttct

SEQ ID NO: 4 - DNA, 20 bp, synthetic construct
  source 1..20: mol_type = other DNA
ctttacttactacacctcag

SEQ ID NO: 5 - DNA, 12 bp, synthetic construct
  source 1..12: mol_type = other DNA
atcgagatttct

(19) United States
(12) Patent Application Publication
(10) Pub. No.: US 2025/0092445 A1
(43) Pub. Date: Mar. 20, 2025

(54) PRIMER SET AND GENE CHIP METHOD FOR DETECTING SINGLE BASE MUTATION

(71) Applicant: MANJING GENSCRIPT BIOTECH CO., LTD., Jiangsu (CN)

(72) Inventors: Feichi HU, Jiangsu (CN); Qi WANG, Jiangsu (CN); Cheng-Hsien WU, Jiangsu (CN)

(21) Appl. No.: 18/293,339

(22) PCT Filed: Aug. 25, 2022
(86) PCT No.: PCT/CN2022/114653

§ 371 Date: Jan. 29, 2024

(30) Foreign Application Priority Data
Aug. 25, 2021 (CN) 202110980219.2

(51) Int. Cl. C12Q 1/6827; C12Q 1/686

(52) U.S. Cl. CPC C12Q 1/6827; C12Q 1/686

(57) ABSTRACT

Provided are a primer set and a gene chip method for detecting a single base mutation, wherein the gene chip method comprises: providing a sample containing a nucleic acid sequence to be detected; amplifying the nucleic acid sequence to be detected in the sample by using a primer set by means of a polymerase chain reaction, and purifying the amplification product obtained after the polymerase chain reaction to remove unreacted primers and enzymes; and hybridizing the purified amplification product with a chip probe, and detecting mutation information of the nucleic acid sequence to be detected by using a chip reader. According to the method, a target nucleic acid sequence is amplified while a base mutation site is identified, which not only achieves the enhanced sensitivity of detection, but also can achieve simultaneous and rapid detection of multiple target nucleic acid sequences on the basis of the characteristics of a high-throughput dot matrix of a chip, high reading speed, etc.

### CROSS REFERENCE TO RELATED APPLICATION

The present application claims priority to Chinese patent application No. 202110980219.2, filed on Aug. 25, 2021, which is incorporated herein by reference in its entirety.

### TECHNICAL FIELD

The present invention belongs to the field of biological detection, and particularly relates to a primer set and a gene chip method for detecting a single base mutation.

### BACKGROUND ART

Gene chips provide a new development direction in the field of in vitro diagnostics due to their characteristics of having high-throughput and being fast and convenient. They have broad application prospects in genetic disease detection, disease screening, disease classification, pathogen detection, personalized medication, etc. In-depth development of gene chip diagnostic applications will effectively strengthen disease diagnosis and treatment and protect human health. However, there are still some challenges during the development. For example, an unduly simple detection method (direct in situ hybridization) is used, and no exquisite design is involved, which limits the detection sensitivity. Especially for the single nucleotide polymorphism (SNP) detection, the traditional in situ hybridization is far from adequate, as it is necessary to ensure accurate mutation site identification while maintaining a sufficiently high detection sensitivity.

To this end, some combined methods or methods with enhanced sensitivity have been developed in this field. For example, the Golden-gate method developed by the world-renowned gene chip company Illumina combines the rolling circle amplification method with in situ hybridization, which can effectively improve the detection sensitivity. However, the method also increases the detection cost and operational complexity (see KL-FCM clustering analysis in Illumina Golden Gate DNA methylation micro-array, Chinese Journal of Bioinformatics. 2014(000) 002). Another method allows the high molecular weight support based on the in situ hybridization method of chips assisted by the cyclic chain reaction, thereby achieving highly sensitive detection (see Biosensors and Bioelectronics 49 (2013) 472-477). However, this method has the disadvantage of an excess of adsorption and contamination induced by excessive in-situ reactions, which likely leads to an increase in the detection baseline or false positive results. There are also methods that allow for the binding on the chips by virtue of nanomaterials or newly synthesized signal-responsive sensitive molecules, thereby achieving the purpose of highly sensitive detection (see Sensors and Actuators B: Chemical, 272, 53-59; Journal of the American Chemical Society, 138(42), 13975-13984). The disadvantages of the methods lie in that the synthesis of the nanomaterials or new molecules is challenging, and the size or purity is difficult to control, resulting in a reduced detection reproducibility of the methods.

All of these methods have certain defects. They either involve unduly simple technique, which limits the detection sensitivity, or improve the detection sensitivity at the sacrifice of increased detection cost and operational complexity and even reduced detection accuracy. Therefore, it is necessary to find an inexpensive and easy-to-operate method for detecting SNP accurately and rapidly.

### SUMMARY OF THE INVENTION

An objective of the present invention is to provide a rapid, accurate and inexpensive method for detecting a single base mutation (also referred to as single nucleotide mutation, single nucleotide typing, single nucleotide polymorphism (SNP)) by overcoming the defects of the existing single base mutation detection method, such as reduced detection sensitivity, and high detection cost and operational complexity.

Based on the previous experience in developing diagnostic methods, the inventors of the present invention develop a novel detection method combining unbalanced PCR with chip capture. The present invention further combines unbalanced PCR with high-throughput and highly sensitive electrochemical chips through ingenious primer design and achieves the simultaneous and quantitative detection of multiple mutation sites of target sequences.

In order to achieve the above-mentioned objective, in an aspect, the present invention provides a primer set for detecting a single base mutation in a nucleic acid sequence, wherein the primer set comprises the following primers:
one or more identifying primers, wherein the identifying primer comprises in order in the 5′ to 3′ direction: a) a nucleotide sequence that can specifically bind to a detection tool; b) a spacer arm molecule that can prevent further PCR amplification; c) a nucleotide sequence that is complementary to a stretch of contiguous nucleotides in the nucleic acid sequence to be detected, wherein the 5′ end of the contiguous nucleotides starts from the first nucleotide downstream of a mutation detection site, and d) a single nucleotide selected from any one of A, T, C, and G, and
an amplification primer, wherein the amplification primer is modified at the 5′ end with a group that can bind to a signal-responsive molecule, and the amplification primer can be used to amplify the amplification product obtained by amplifying the nucleic acid sequence to be detected using the identifying primer.

In the context of the present description, the “nucleic acid” can be double-stranded or single-stranded nucleic acid, such as double-stranded DNA, single-stranded DNA or RNA.

In the context of the present description, the “single base mutation” refers to a mutation generated by the replacement of a single base in a nucleic acid sequence.

For the primer set for single nucleotide mutation detection of the present invention, two primers with different amplification functions (and in general, different lengths) are designed. One of the primers is referred to as an identifying primer and the other is referred to as an amplification primer. In addition, in view of the difference in the length of the nucleotide sequence contained in the primers that is specifically complementary to the template during PCR amplification, the identifying primer comprises a short-stranded primer that is specifically complementary to the nucleic acid sequence to be detected, and the amplification primer is a long-stranded primer comprising a group that can bind to a signal-responsive molecule.

For example, in an embodiment of the present invention, the nucleotide sequence contained in the identifying primer that is specifically complementary to the sequence to be detected (target sequence) may be 1 to 19, particularly 3 to 15 (such as 3, 4, 5, 6, 7, 8, 9, 10, 11, 12, 13, 14 or 15) nucleotides shorter than that of the amplification primer in length. In some preferred embodiments, the nucleotide sequence contained in the identifying primer that is specifically complementary to the sequence to be detected (target sequence) may be 2 to 8, such as 2, 3, 4, 5, 6, 7 or 8 nucleotides shorter than that of the amplification primer in length. More particularly, portions c) and d) of the identifying primer may be between 11 and 16 nucleotides (such as 11, 12, 13, 14, 15 or 16 nucleotides) in total length; and the amplification primer may be between 15 and 30 nucleotides (such as 15, 16, 17, 18, 19, 20, 21, 22, 23, 24, 25, 26, 27, 28, 29 or 30 nucleotides) in length. In some preferred embodiments, portions c) and d) of the identifying primer are 12 to 15 nucleotides in total length. In a specific embodiment, portions c) and d) of the identifying primer are 12 nucleotides in total length. In a preferred embodiment, the amplification primer is 16 to 20 nucleotides in length. In a specific embodiment, the amplification primer is 20 nucleotides in length.

In the context of the present description, the term “identifying primer” refers to a primer comprising a sequence that is specifically complementary to and binds to a probe, a spacer arm molecule and a sequence that is specifically complementary to and binds to a target sequence, wherein the nucleotide of the primer at the 3′ end may be selected from any one of A, T, C, and G according to the detection purpose. The term “short-stranded primer” refers to a primer that comprises a sequence that is specifically complementary to and binds to a target sequence, wherein the nucleotide of the primer at the 3′ end may be selected from any one of A, T, C, and G according to the detection purpose. In this application, the identifying primer comprises a short-stranded primer, and the short-stranded primer sequence is a critical sequence for functioning to identify the target sequence. The term “amplification primer” refers to a long-stranded primer comprising a group that can bind to a signal-responsive molecule, for example, a long-stranded primer being modified at the 5′ end with a group that can bind to a signal-responsive molecule. The term “long-stranded primer” refers to a primer can be used to amplify the amplification product obtained by amplifying the target sequence using the identifying primer or short-stranded primer.

Based on the unique design of the primer set of the present invention, the PCR performed using the primers of the present invention is also referred to as unbalanced PCR. Particularly, when the nucleotide (i.e., portion d)) at the 3′ end of the short-stranded primer cannot achieve correct pairing with the nucleotide at the single nucleotide mutation site of the nucleic acid sequence to be detected (target nucleic acid sequence), the binding force between the short-stranded primer and the target nucleic acid sequence is greatly weakened and can hardly support the hybridization, resulting in no amplification product; and when the nucleotide at the 3′ end of the short-stranded primer achieves correct pairing with the nucleotide at the single base mutation site of the target nucleic acid sequence, a double-stranded amplification product can be obtained from the amplification at a suitable annealing temperature. The amplification products obtained with the long-stranded primer and the short-stranded primer have a strong hybridization force and thus can bind to each other, thereby achieving the replication of the amplification product of the short-stranded primer. After enlargement through multiple PCR cycles, the correct specific amplification products generated from the long-stranded primer and the short-stranded primer are exponentially amplified.

Further, each short-stranded primer will first be modified with a spacer arm molecule at the 5′ end and then the spacer arm molecule is linked to a nucleotide sequence that can specifically bind to a detection tool, wherein the spacer arm molecule functions to prevent further PCR amplification and thus guarantees that portion a) of the identifying primer is in a single-stranded state; and the long-stranded primer will be modified with a group that can bind to a signal-responsive molecule at the 5′ end.

More particularly, the detection tool can be any common detection tool in the art that can be used to detect nucleotide sequences. For example, the detection tool can be a chip modified with a gene probe, an electrochemical detection electrode modified with a gene probe, or a fluorescent detection nanomaterial modified with a gene probe. The chip modified with a gene probe can be obtained using common methods in the field, and the chip can be, for example, a metal chip, a semiconductor chip, a glass chip, a paper chip, etc. The electrochemical detection electrode modified with a gene probe can be obtained by linking an oligonucleotide fragment on an electrode (such as a platinum electrode) via sulfhydryl, or by direct on-chip growing and synthesis. The fluorescent detection nanomaterials modified with a gene probe can be a metal nanomaterial, silicon, silica, or a carbon nanomaterial, and can be obtained by treating the surface of a nanomaterial with a silanizing reagent to generate an amino group and linking same to an oligonucleotide fragment that is modified with a carboxyl group.

According to the present invention, the spacer arm molecule can be any molecule that can prevent further amplification of the sequence amplified from the short-stranded primer. For example, the spacer arm molecule can be a molecule with a length of not less than 5 carbon-carbon bonds, carbon-oxygen bonds or carbon-nitrogen bonds, etc., and is not a deoxyribonucleotide molecule. In some embodiments, the spacer arm molecule can be a non-deoxyribonucleotide molecule that has a backbone with a length of not less than 5 carbon-carbon bonds. In some embodiments, the spacer arm molecule can be a hydrocarbon chain molecule that has a backbone with a length of not less than 5 carbon-carbon bonds. For example, the backbone can be a hydrocarbon chain molecule having 6 carbon atoms, 9 carbon atoms or 12 carbon-atoms, etc. (i.e., C6, C9 or C12).

More particularly, the group that can bind to a signal-responsive molecule can be selected based on the experience of those skilled in the art and actual needs, and any chemical molecule or biomolecule group that can react with and bind to a signal-responsive molecule in an aqueous phase at room temperature suits the purpose. For example, the group that can bind to a signal-responsive molecule may be biotin, phenylboronic acid, mannose, an antigen, an antibody, etc. The signal-responsive molecule and the group that can bind to a signal-responsive molecule refer to any pair of molecules that can link or bind to each other through a chemical reaction, wherein the signal-responsive molecule comprises an electrochemical substance or a chromogenic substance. For example, correspondingly, the signal-responsive molecule can be streptavidin, a sugar molecule with a hydroxyl group at the ortho position, canavalin, an antibody, an antigen, etc., which is linked to electrochemical substances such as horseradish peroxidase and alkaline phosphatase or linked to chromogenic substances such as fluorescein, fluorescent proteins or acridine compounds. In some embodiments, an electrochemical signal detection can be performed after the termination of a reaction between a reaction substrate added (such as TMB) and a signal-responsive molecule (such as horseradish peroxidase-labeled streptavidin), which has been bound to the group (such as biotin) that can bind to the signal-responsive molecule and modifies the amplification primer.

Further, when correct pairing is achieved as described above, portion a), as a single strand, that can specifically bind to a detection tool and the group that can bind to a signal-responsive molecule will be introduced into the double-stranded product, wherein portion a), as a single strand, specifically binds to the detection tool (such as a probe chip) and thus can be immobilized on the chip; and the group that can bind to a signal-responsive molecule can bind to a signal-responsive molecule linked to an electrochemical substance or a chromogenic substance to generate a detection signal, which facilitates subsequent chip capture and detection.

In addition, the primer set of the present invention may comprise one or more identifying primers as required by the detection. For example, when the detection method of the present invention is used to detect whether the nucleotide at the expected single base mutation site of the target nucleic acid sequence is mutated, an identifying primer whose base at the 3′ end is complementary to the base at the expected mutation site of the unmutated wild-type nucleic acid sequence can be used for detection. As another example, when the detection method of the present invention is used to determine the type of the base at the single base mutation site of the target nucleic acid sequence where the base mutation occurs, one or more (e.g., two, three, or four) identifying primers whose bases at the 3′ end can be any one of A, T, C and G can be used for detection. In some embodiments, when the method of the present invention is used to detect whether mutation occurs or there is an expected mutant nucleotide at the expected single base mutation site of the nucleic acid sequence to be detected, portion d) of the identifying primer is a nucleotide complementary to the unmutated nucleotide or the expected mutant nucleotide at the expected single base mutation site of the nucleic acid sequence to be detected.

The unbalanced PCR reaction allows for the amplification of the target nucleic acid sequence while identifying the single nucleotide (base) mutation site and the amplification product facilitates subsequent chip capture and detection, thereby achieving the enhanced sensitivity of the detection. In addition, on the basis of the characteristics of a high-throughput dot matrix of a chip, high reading speed, etc., the method can achieve simultaneous and rapid detection of multiple target nucleic acid sequences. It can be seen therefrom that the method of the present invention has the characteristics of achieving high-throughput, highly sensitive, and rapid detection.

In another aspect, the present invention further provides a gene chip method for detecting a single base mutation in a nucleic acid sequence, wherein the method comprises:
1) providing a sample containing a nucleic acid sequence to be detected;
2) amplifying the nucleic acid sequence to be detected in the sample by using the primer set as described above by means of a polymerase chain reaction;
3) purifying the amplification product obtained after the polymerase chain reaction to remove unreacted primers and enzymes; and
4) hybridizing the purified amplification product with a chip probe, and detecting mutation information of the nucleic acid sequence to be detected by using a chip reader.

According to the present invention, the gene chip method for detecting a single base mutation in a nucleic acid sequence is also referred to as unbalanced PCR chip method, which mainly includes three parts: unbalanced PCR, purification, and chip capture and detection. FIG. 1 schematically shows a flow chart of the unbalanced PCR chip method of the present invention.

### Unbalanced PCR

This part involves using the primer set of the present invention to amplify a target nucleic acid sequence by means of unbalanced PCR. The features of the primer set used are the same as described above. To avoid unnecessary redundancy, the features of the primer set will not be repeated here.

The amplification reaction (i.e., polymerase chain reaction) in the method of the present invention can be performed in an amplification reaction mixture. The mixture comprises the reagents required to complete a primer extension reaction or nucleic acid amplification. Non-limiting examples of such reagents comprise primers, polymerases, buffers, cofactors (e.g., divalent or monovalent cations), and nucleotides (e.g., dNTPs).

In the present invention, the polymerase chain reaction is performed using a DNA polymerase. The DNA polymerase may be a common DNA polymerase known in the art. In some embodiments, the DNA polymerase is a high-fidelity polymerase. In some embodiments, the DNA polymerase is selected from: Hot-Start Taq polymerase (E00049, GenScript Biotech Corporation), TaqNova Stoffel DNA polymerase (RP810, BLIRT), HiFi-KAPA polymerase 2× (KK2601, Roche), Hemo KlenTaq polymerase (M0332S, NEB), etc. In a preferred embodiment, the DNA polymerase is HiFi-KAPA polymerase.

In some embodiments of the present invention, the polymerase chain reaction may include a pre-denaturation step, a cyclic amplification step and a final extension step, wherein the cyclic amplification step may include denaturation, annealing and extension steps in each cycle. In some embodiments, 18-30 cycles, such as 20 cycles, are performed in the cyclic amplification step. In some embodiments, each cycle in the cyclic amplification step is performed under the following conditions: 98° C., 10 s: 45-52° C., 15-30 s; and 72° C., 15 s. In some embodiments, the annealing temperature is 44° C. to 52° C., such as 44° C., 45° C., 46° C., 47° C., 48° C., 49° C., 50° C., 51° C., or 52° C., preferably 45° C.-50° C. In a specific embodiment, the annealing temperature is 45° C. In another specific embodiment, the annealing temperature is 50° C.

### Purification

The purification process of the present invention can be carried out using column purification or magnetic beads with the aim to remove unreacted primers and enzymes and obtain relatively pure double-stranded DNA target products. Taking magnetic bead purification as an example, the purification using smart beads (Wuhan Yeasen Biotechnology Co., Ltd.) comprises the following operations according to the instructions provided by the manufacturer: 1) the magnetic beads are taken out of the refrigerator and equilibrated at room temperature for at least 30 minutes; 2) the magnetic beads are subject to vortex shaking or thoroughly inverted to allow uniform mixing; 3) Hieff NGS® Smarter DNA Clean Beads of the same volume (1.0×, × represents the volume of the DNA sample to be purified) are taken and added into the DNA solution (in EP tube with PCR products), and the mixture are incubated at room temperature for 5 minutes; 4) after brief centrifugation, the PCR tube is placed on a magnetic stand to separate the magnetic beads and liquid, and after the solution is clear (about 5 minutes), the supernatant is carefully removed; 5) while the PCR tube is kept on the magnetic stand, 200 μL of a freshly prepared 80% ethanol solution is added to rinse the magnetic beads, and after incubation at room temperature for 30 seconds, the supernatant is carefully removed; 6) step 5 is repeated, and the tube is rinsed twice in total; 7) while the PCR tube is kept on the magnetic stand, the lid is opened and the magnetic beads are air-dried until cracks just appear (about 5 minutes); 8) the PCR tube is removed from the magnetic stand, 21 μL of ddH₂O is added, and the mixture is gently pipetted with a pipette to allow uniform mixing and is allowed to stand at room temperature for 5 minutes; 9) the PCR tube is briefly centrifuged and placed on the magnetic stand, after the solution is clear (about 5 minutes), 20 μL of the supernatant is carefully transferred into a new PCR tube while the magnetic beads are not touched, and then pure double-stranded DNA products can be obtained.

### Chip Capture and Detection

PBST buffer can be added to the purified PCR products until the total volume reaches 50 uL, and then the mixed solution is added onto a chip surface and the mixture is reacted at 45° C. for 40 minutes. Various probe sequences that can hybridize with the PCR products have been synthesized on the chip in advance. The probe sequences mainly hybridize with the specific end sequences of different primers. Since the long-stranded primer is modified with a group that can bind to an electrochemical signal molecule, signal molecules are linked to the top end of the double strands upon reaction at room temperature for 20 minutes. The chip is placed in a chip reader, i.e., electron capture detector (ECD), and the signal of each dot can be read. Upon analysis of the dot matrix signals (distribution or strength) on the chip, the mutation information of the target sequence (including, e.g., amount of mutated sequences, mutation type, and mutation position) can be acquired.

The present invention further comprises a kit for detecting a single base mutation in a nucleic acid sequence, the kit comprising the primer set as described above. In a preferred embodiment, the kit may also comprise a DNA polymerase, preferably a high-fidelity polymerase.

In addition, the present invention provides the use of the above-mentioned primer set or kit in the detection of a single base mutation in a nucleic acid sequence, particularly the use of the above-mentioned primer set or kit in a gene chip method for detecting a single base mutation in a nucleic acid sequence.

### BRIEF DESCRIPTION OF THE DRAWINGS

The accompanying drawings are used for providing a further understanding of the present invention, constitute a part of the description, and are used, together with the following specific embodiments, for explaining the present invention but do not limit the present invention. In the accompanying drawings:

FIG. 1 schematically shows a flow chart of the unbalanced PCR chip method of the present invention.

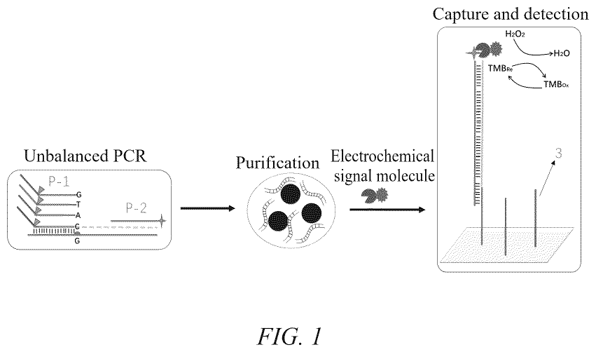

FIG. 2 shows the gel electrophoresis image of the wild type and mutant (single base mutation) target sequences after unbalanced PCR amplification is performed, wherein lane 1 shows the mutant target sequence, lane 2 shows the wild type sequence, and lane 3 is a blank control using water in place of the target sequence. The leftmost and rightmost lanes are molecular weight markers (the molecular weights from top to bottom are 3000, 2000, 1500, 1000, 700, 500, 250, and 100 bp).

FIG. 3 shows the gel electrophoresis image of the mutant target sequences after PCR amplification is performed using primers p-1-c9 and p-1. Lane 1 is the gel electrophoresis image of the product after PCR is performed on the target sequence using the primer p-1-c9, which is modified with a spacer arm molecule and has a sequence complementary to the chip probe. Lane 2 is the gel electrophoresis image of the product after PCR is performed on the target sequence using the primer p-1, which is not modified with a spacer arm molecule and has no identification sequence complementary to the chip probe. Lane 3 is the molecular weight marker (the molecular weights from top to bottom are 3000, 2000, 1500, 1000, 700, 500, 250, and 100 bp).

FIG. 4 shows the results of chip hybridization detection of the PCR products, wherein a is the electrochemical image obtained by using the chip reader ECD to detect the four products after their hybridization and capture on the chip surface; and b shows the measured electrochemical signals of the target dot matrix collected from the chip (the abscissa shows the corresponding dot matrix probe, and the ordinate shows the average value of the net increment of the signal).

FIG. 5 shows the detection results obtained from unbalanced PCR combined with chip hybridization at a low concentration. FIG. 5a is the gel electrophoresis image of the target sequences at gradient concentrations after the first step (unbalanced PCR); FIG. 5b is the electrochemical image obtained after the chip is hybridized with the purified PCR products and then placed into the chip reader ECD; and FIG. 5c shows the specific signal values corresponding to FIG. 5b, i.e., the measured electrochemical signal increment of the chip, wherein the abscissa shows the PCR products at different concentrations, and the ordinate shows the average value of the net increment of the signal.

### DETAILED DESCRIPTION OF EMBODIMENTS

The specific embodiments of the present invention are described in detail below. It should be understood that the specific embodiments described herein are merely used to illustrate and explain the present invention, but not intended to limit the present invention.

The endpoints of ranges and any values disclosed herein are not limited to the precise range or values, but these ranges or values are to be construed as including values close to such ranges or values. For numerical ranges, one or more new numerical ranges can be obtained by combining the endpoint values of various ranges, combining the endpoint values of various ranges and individual point values, and combining individual point values. These value ranges shall be deemed to be specifically disclosed herein.

The embodiments of the present invention are illustrated below by using the specific examples, and those skilled in the art would have readily understood other advantages and effects of the present invention from the disclosure of the present description, and it is obvious that the described examples are some of the examples of the present invention rather than all the examples. On the basis of the embodiments of the present invention, all other embodiments obtained by those of ordinary skill in the art without involving any inventive effort fall within the scope of protection of the present invention.

### EXAMPLES

### Example 1: SNP Detection with Unbalanced PCR Primers (Specific Detection)

### Mutant Target Sequence

(SEQ ID NO: 1)
5'-CTTTACTTACTACACCTCAGATATATTTCTTCATGAA
GACCTCACAGTAAAAATAGGTGATTTTGGTCTAGCTACAG
AAGAAATCTCGATGGAGTGGG

### Wild-Type Sequence

(SEQ ID NO: 2)
5'-CTTTACTTACTACACCTCAGATATATTTCTTCATGAA
GACCTCACAGTAAAAATAGGTGATTTTGGTCTAGCTACAG
ATGAAATCTCGATGGAGTGGG

Unbalanced PCR primers are designed and can be seen in Table 1 below. The identifying primer p-1-c9 comprises three portions: the 5′ end portion is the sequence (CAGCTAGAGCTCCAGT, 16 nt, SEQ ID NO:7) that can be specifically complementary to and bind to the chip probe; the middle portion is a specially designed modifying spacer arm molecule C9, which functions to block further PCR amplification, and thus guarantees that the 5′ end portion is in a single-stranded state and can be further complementary to and hybridize with the chip probe; and the 3′ end portion is the PCR short-stranded primer portion that can specifically bind to the target sequence. Long-stranded primer p-2 is designed. The primer is modified at the 5′ end with a group (biotin) that can bind to a signal-responsive molecule.

TABLE 1

| Primer name | Sequence |
|---|---|
| p-1-c9 | CAGCTAGAGCTCCAGT(spacer) |
| | ATCGAGATTTCT (SEQ ID NO: 3), |
| | the spacer is C9 |
| p-2 | Biotin-CTTTACTTACTACACCTCAG |
| | (SEQ ID NO: 4) |

The amount of the wild-type or mutant nucleotide sequence template was 1 nmol·L⁻¹ and the PCR reaction was performed. The PCR reaction was performed on a Biometra T1 thermolcycler using the following specific reaction system and reaction conditions. Reaction system: primer p-1-c9 (10 μmol·L⁻¹) 1 μL, primer p-2 (10 μmol·L⁻¹) 1 μL, template DNA (1 nmol·L⁻¹) 1 μL, 2× HiFi-KAPA polymerase (KK2601, Roche) 10 μL, and ddH₂O making up to 20 μL. Reaction conditions: pre-denaturation at 98° C. for 30 s; denaturation at 98° C. for 10 s, annealing at 50° C. for 30 s, extension at 72° C. for 15 s, 20 cycles; and extension at 72° C. for 5 min. The same PCR reaction using water as the template was performed as a blank control.

After the reaction, the products were analyzed by agarose gel electrophoresis: 2 μL of each of the above-mentioned PCR products were taken and mixed evenly with 0.5 μL of a plasmid maxi extraction dye (Goldview, SBS Genetech Co., Ltd.). The mixed solutions were added into the wells with a 2.5% agarose gel (Invitrogen), the gel electrophoresis was carried out, and the gel electrophoresis images were collected (model DYY-8C, Beijing Liuyi Biotechnology Co., Ltd.; 120 V, 20 min).

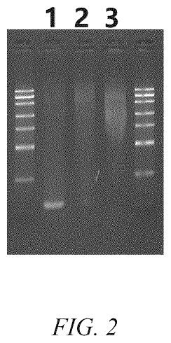

The results are as shown in FIG. 2. Only the mutant target sequence (lane 1) shows an obvious band, whereas the wild-type sequence and blank control (lanes 2 and 3, respectively) do not show bands. Theoretically, for the wild-type sequence and water, no reaction was carried out so that no bands were shown. The results are consistent with the theory and demonstrate that this method can be used to accurately identify single base mutations.

### Example 2: Verification of Spacer Arm-Containing Primer for Single Base Mutation Detection

The target sequence to be detected is the mutant target sequence

(SEQ ID NO: 1)
5'-CTTTACTTACTACACCTCAGATATATTTCTTCATGAAG
ACCTCACAGTAAAAATAGGTGATTTTGGTCTAGCTACAGAA
GAAATCTCGATGGAGTGGG

The identifying primer p-1-c9 is designed. The primer comprises three portions: the 5′ end portion is the sequence (CAGCTAGAGCTCCAGT, 16 nt) that can be specifically complementary to and bind to the chip probe; the middle portion is a specially designed modifying spacer arm molecule C9, which functions to block further PCR amplification, and thus guarantees that the 5′ end portion is in a single-stranded state and can be further complementary to and hybridize with the chip probe; and the 3′ end portion is the PCR short-stranded primer portion that can specifically bind to the target sequence.

Design of short-stranded primer p-1: only the 3′ end portion of p-1-c9 is retained, which portion can specifically bind to the target sequence and serve as a short-stranded primer for PCR.

Design of long-stranded primer p-2: the primer is modified at the 5′ end with a group (biotin) that can bind to a signal-responsive molecule. The specific sequences can be seen in Table 2 below:

TABLE 2

| Primer name | Sequence |
|---|---|
| p-1-c9 | CAGCTAGAGCTCCAGT(spacer) |
| | ATCGAGATTTCT (SEQ ID NO: 3) |
| p-1 | ATCGAGATTTCT (SEQ ID NO: 5) |
| p-2 | Biotin-CTTTACTTACTACACCTCAG |
| | (SEQ ID NO: 4) |

The templates of the synthesized mutant target sequences were at the concentration of 1 nmol·L⁻¹. PCR reaction system (20 μL) for lane 1: primer p-1-c9 (10 μmol·L⁻¹) 1 μL, primer p-2 (10 μmol·L⁻¹) 1 μL, template DNA (1 nmol·L⁻¹) 1 μL, and 2× HiFi-KAPA polymerase (KK2601, Roche) 10 μL.

PCR reaction system for lane 2: primer p-1 (10 μmol. L⁻¹) 1 μL, primer p-2 (10 μmol·L⁻¹) 1 μL, template DNA (1 nmol·L⁻¹) 1 μL, 2× HiFi-KAPA polymerase (KK2601, Roche) 10 μL, and ddH₂O making up to 20 μL. PCR reaction conditions: pre-denaturation at 98° C. for 30 s; denaturation at 98° C. for 10 s, annealing at 50° C. for 30 s, extension at 72° C. for 15 s, 20 cycles; and extension at 72° C. for 5 min.

After the reaction, the products were analyzed by agarose gel electrophoresis: 2 μL of each of the above-mentioned corresponding PCR products were taken and mixed evenly with 0.5 μL of a plasmid maxi extraction dye (Goldview, SBS Genetech Co., Ltd.). The two mixed solutions were added into the wells of the respective lanes with a 2.5% agarose gel (Invitrogen), the gel electrophoresis was carried out, and the gel electrophoresis images were collected (model DYY-8C, Beijing Liuyi Biotechnology Co., Ltd.; 120 V, 20 min).

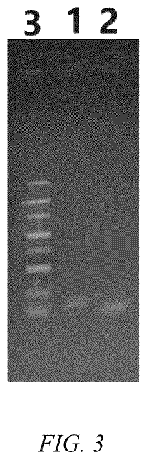

The results are shown in FIG. 3. Bright target bands can be seen in all lanes, and the position of the band in lane 1 in the figure is slightly higher than that of the band in lane 2. This is because the product, obtained in the PCR using the primer modified with a spacer arm and a hybridization strand, has a slightly larger molecular weight than the product obtained in the PCR using the unmodified primer. The results are consistent with the theory and demonstrate that the spacer arm and the chip hybridization sequence has been successfully introduced into the PCR product.

### Example 3: PCR Product Purification

The three PCR products obtained in Example 1 were purified using magnetic beads (smart beads, Wuhan Yeasen Biotechnology Co., Ltd.), and the following operations were carried out according to the instructions provided by the manufacturer: the magnetic beads were taken out of the refrigerator and equilibrated at room temperature for at least 30 minutes and then subject to vortex shaking or thoroughly inverted to allow uniform mixing. Hieff NGS® Smarter DNA Clean Beads of the same volume (1.0×, × represents the volume of the DNA sample to be purified) were taken and added into the DNA solution (in EP tube with PCR products), and the mixture were incubated at room temperature for 5 minutes. After brief centrifugation, the PCR tube was placed on a magnetic stand to separate the magnetic beads and liquid. After the solution was clear (about 5 minutes), the supernatant was carefully removed. While the PCR tube was kept on the magnetic stand, 200 μL of a freshly prepared 80% ethanol solution was added to rinse the magnetic beads, and after incubation at room temperature for 30 seconds, the supernatant was carefully removed. Step 5 was repeated, and the tube was rinsed twice in total. While the PCR tube was kept on the magnetic stand, the lid was opened and the magnetic beads were air-dried until cracks just appeared (about 5 minutes). The PCR tube was removed from the magnetic stand, 21 μL of ddH₂O was added, and the mixture was gently pipetted with a pipette to allow uniform mixing and was allowed to stand at room temperature for 5 minutes. The PCR tube was briefly centrifuged and placed on the magnetic stand. After the solution was clear (about 5 minutes), 20 μL of the supernatant was carefully transferred into a new PCR tube while the magnetic beads were not touched. Then pure double-stranded DNA products were obtained.

The concentrations of the three purified products were detected with Thermo Scientific™ NanoDrop™ One Microvolume UV-Vis Spectrophotometers. The concentrations of the above-mentioned purified products were measured and are shown in Table 3. The concentration of sample 1 (PCR products of the mutant target sequence) is much higher than those of samples 2 and 3 (PCR products of the wild-type sequence and blank control, respectively), which proves that unbalanced PCR based on primers of different lengths can be used to effectively identify and amplify the single base mutation of the mutant target sequence, and the products were successfully purified.

TABLE 3

| Sample | 1 | 2 | 3 |
|---|---|---|---|
| Concentration | 15.8 | 6.0 | 4.2 |
| (ng/uL) | | | |

### Example 4: Unbalanced PCR Combined with Chip Hybridization for PCR Product Detection

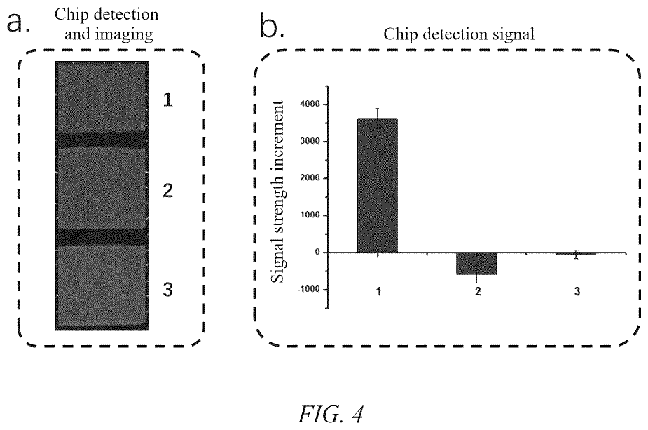

PBST buffer was added to the three purified PCR products from Example 3 until the total volume reached 50 μL, and then the mixtures were added to three cavities of the chip (the chip was artificially divided into four non-interfering areas and the upper three areas were used in this experiment) for hybridization as shown in FIG. 4. The mixtures were reacted at 45° C. for 40 minutes. The chip had been linked to probe sequences (ACTGGAGCTCTAGCTGTTTTTTTTTT, SEQ ID NO: 6) that can hybridize with and bind to the PCR products. The probe sequences were used for specifically hybridizing with the amplification products obtained in the PCR using primers p-1-c9/p-2. The chip was designed to have three rectangular areas representing the three areas with each being a long strip shape of different widths as shown in FIG. 4a. There were other control probes on the chip dot matrix that do not hybridize with the purified PCR products and are used to detect interference signals in the experiment.

Primer p-2 was modified at a site with a group (biotin) that can bind to an electrochemical signal molecule. Therefore, the amplified PCR products also had the binding site. Upon reaction at room temperature for 20 minutes, the signal molecule (HRP-labeled Streptavidin (SA-HRP)) was linked at the binding site on the top end of the double strand of the PCR product. The chip was placed in the chip reader ECD, and the signal of each dot can be read. The results are shown in FIG. 4. After the three products were hybridized and captured on the chip surface, the electrochemical images were obtained with the chip reader ECD. Upon comparison of the three areas in FIG. 4a, it can be found that after the addition of the target products, only the gray scale values of the matched probes increase significantly in the first area, while the gray scale values of other matrix dots are equivalent to the background, demonstrating that by the method, the products of the target sequence were successfully detected without obvious interference signals. Areas 2 and 3 on the chip show the results of the wild-type sequence and blank (water). It can be found that gray scale values do not increase in all dot matrices (including the target dot matrix and blank dot matrix), demonstrating that double-stranded products cannot be generated from the wild-type sequence by amplification with the designed primers, which is consistent with the theory, and also demonstrating that this method can be used to correctly identify the single base mutation of the mutant target sequence. FIG. 4b shows the measured electrochemical signals of the target dot matrix collected from the chip (the abscissa shows the corresponding dot matrix probe, and the ordinate shows the average value of the net increment of the signal). It can be found that obvious signal increment can be seen with only the target dot matrix probes in the first area, and barely any obvious signal can be seen with the dot matrix probes of the other areas, which corresponds to the image in FIG. 4a and is consistent with the theory. The purpose of quantitative detection of SNP can also be achieved by virtue of the specific signals in FIG. 4b.

### Example 5: Detection of Products at Different Low Concentrations

The target sequence to be detected is the (mutant) target sequence

(SEQ ID NO: 1)
5'-CTTTACTTACTACACCTCAGATATATTTCTTCATGA
AGACCTCACAGTAAAAATAGGTGATTTTGGTCTAGCTAC
AGAAGAAATCTCGATGGAGTGGG

The synthesized mutant target sequence was at the concentration of 1 nmol·L⁻¹. The mutant target sequence was diluted with TE buffer to 1 pM, 100 fM, 10 fM, and 1 fM through gradient dilution respectively. PCR amplification was performed using the mutant target sequence at different concentrations as the amplification templates. Amplification system: primer p-1-c9 (10 μmol·L⁻¹) 1 μL, primer p-2 (10 μmol·L⁻¹) 1 μL, templates at different concentrations 1 μL, 2× HiFi-KAPA polymerase (KK2601, Roche) 10 μL, and ddH₂O making up to 20 μL. Reaction conditions: pre-denaturation at 98° C. for 30 s; denaturation at 98° C. for 10 s, annealing at 50° C. for 30 s, extension at 72° C. for 15 s, 20 cycles; and extension at 72° C. for 5 min.

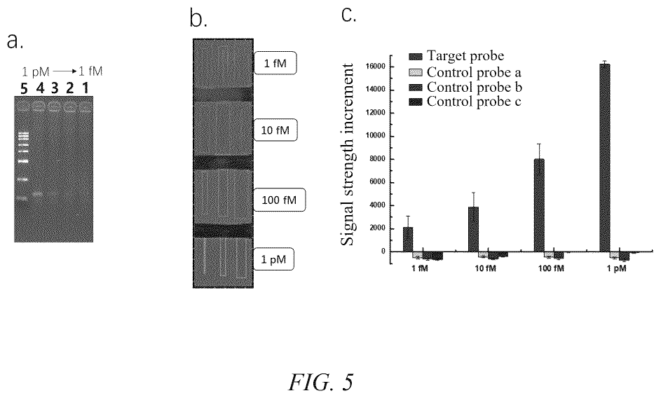

2 μL of sample from each of the PCR products was taken and mixed evenly with 0.5 μL of a plasmid maxi extraction dye. The mixed solutions were added into the wells with a 2.5% agarose gel and the gel was run on an electrophoresis instrument. After the running of the gel was completed, the results were observed under a UV lamp. As shown in FIG. 5a, the template amounts are different and are as follows from right to left: template 1:1 fM, template 2:10 fM, template 3:100 fM, template 4:1 pM, and template 5: ladder (molecular weights from top to bottom: 3000, 2000, 1500, 1000, 700, 500, 250, and 100 bp). It can be found that as the concentration decreases, the brightness of the bands gradually become darker, demonstrating that the amplification products were gradually reduced, which was consistent with the theory. FIG. 5b is the electrochemical image obtained after the chip is hybridized with the purified PCR products and then placed into the chip reader ECD. The experimental operations are as described in the portion of chip hybridization and detection in Example 4, wherein the electrochemical signal molecule for the analysis can also be replaced with higher amount of SA-poly-HRP molecule, so as to further improve the sensitivity. It can be seen that in the four areas from top to bottom on the chip, PCR products at different concentrations are hybridized respectively. The corresponding matrix dots gradually become brighter and the gray scale values gradually increase, while the gray scales of the control matrix dots do not increase obviously, demonstrating that the target objects are accurately detected on the chip. The results are consistent with the theory. FIG. 5c shows the specific signal values corresponding to FIG. 5b, i.e., the measured electrochemical signal increment of the chip, wherein the abscissa shows the PCR products at different concentrations, and the ordinate shows the average value of the net increment of the signal. As the concentration increases, the electrochemical signal increment also increases correspondingly. The signals of the control matrix dots do not have obvious change, which corresponds to the image in FIG. 5b and is consistent with the theory. The results demonstrate that this method is feasible and can be used to detect the target sequence at an ultra-low concentration (<1 fM).

The foregoing describes preferred embodiments of the present invention in detail. However, the present invention is not limited to specific details of the above embodiments. A plurality of simple variations may be made to the technical solutions of the present invention within the scope of the technical concept of the present invention. These simple variations all fall within the scope of protection of the present invention.

In addition, it should be noted that various specific technical features described in the above specific embodiments can be combined in any suitable manner without contradiction. In order to avoid unnecessary repetition, various possible combination manners will not be separately described in the present invention.

In addition, various embodiments of the present invention may be combined at random without deviating from the idea of the present invention, and the combined embodiments also should be deemed as the content disclosed in the present invention.

What is claimed is:

1. A primer set for detecting a single base mutation in a nucleic acid sequence, wherein the primer set comprises the following primers:
one or more identifying primers, wherein the identifying primer comprises in order in the 5′ to 3′ direction: a) a nucleotide sequence that can specifically bind to a detection tool; b) a spacer arm molecule that can prevent further PCR amplification; c) a nucleotide sequence that is complementary to a stretch of contiguous nucleotides in the nucleic acid sequence to be detected, wherein the 5′ end of the contiguous nucleotides starts from the first nucleotide downstream of a mutation detection site, and d) a single nucleotide selected from any one of A, T, C, and G, and
an amplification primer, wherein the amplification primer is modified at the 5′ end with a group that can bind to a signal-responsive molecule, and the amplification primer can be used to amplify the amplification product obtained by amplifying the nucleic acid sequence to be detected using the identifying primer.

2. The primer set according to claim 1, wherein the detection tool is a chip, an electrochemical detection electrode or a fluorescent detection nanomaterial, all of which are modified with a gene probe.

3. The primer set according to claim 1, wherein the spacer arm molecule is a molecule that has a backbone with a length of not less than 5 carbon-carbon bonds, carbon-oxygen bonds or carbon-nitrogen bonds but is not a deoxyribonucleotide molecule, and is preferably a hydrocarbon chain molecule that has a backbone with a length of not less than 5 carbon-carbon bonds.

4. The primer set according to claim 1, wherein the group that can bind to a signal-responsive molecule is a chemical molecule or a biomolecule that can bind to a signal-responsive molecule in an aqueous phase, preferably biotin, phenylboronic acid, mannose, an antigen or an antibody.

5. The primer set according to claim 1, wherein portions c) and d) of the identifying primer are in total 1 to 19 nucleotides shorter than the amplification primer in length.

6. The primer set according to claim 1, wherein portions c) and d) of the identifying primer are between 11 and 16 nucleotides in total length.

7. The primer set according to claim 1, wherein the amplification primer is between 15 and 30 nucleotides in length.

8. A gene chip method for detecting a single base mutation in a nucleic acid sequence, wherein the method comprises:
1. providing a sample containing a nucleic acid sequence to be detected;
2. amplifying the nucleic acid sequence to be detected in the sample by using the primer set according to claim 1 by means of a polymerase chain reaction,
3) purifying the amplification product obtained after the polymerase chain reaction to remove unreacted primers and enzymes; and
4. hybridizing the purified amplification product with a chip probe, and detecting mutation information of the nucleic acid sequence to be detected by using a chip reader.

9. The method according to claim 8, wherein portion d) of the identifying primer is a nucleotide complementary to the unmutated nucleotide or the expected mutant nucleotide at the expected single base mutation site of the nucleic acid sequence to be detected.

10. The method according to claim 8, wherein the polymerase chain reaction is performed using a DNA polymerase, preferably a high-fidelity polymerase.

11. The method according to claim 10, wherein the DNA polymerase is selected from: Hot-Start Taq polymerase, TaqNova Stoffel DNA polymerase, HiFi-KAPA polymerase, and Hemo KlenTaq polymerase.

12. The method according to claim 8, wherein the annealing temperature of the polymerase chain reaction is from 44° C. to 52° C.

13. The method according to claim 8, wherein the purification is column purification or magnetic bead purification.

14. The method according to claim 8, wherein the mutation information of the nucleic acid sequence comprises at least one of the amount of mutated sequences, mutation type, and mutation position.

15. A kit for detecting a single base mutation in a nucleic acid sequence, the kit comprising the primer set according to claim 1.

16. The kit according to claim 15, further comprising a DNA polymerase, preferably a high-fidelity polymerase.

17. Use of the primer set according to claim 1, or the kit according to claim 15 in a gene chip method for detecting a single base mutation in a nucleic acid sequence.

18. The primer set according to claim 3, wherein portions c) and d) of the identifying primer are in total 1 to 19 nucleotides shorter than the amplification primer in length.

19. The primer set according to claim 6, wherein the amplification primer is between 15 and 30 nucleotides in length.

20. The method according to claim 11, wherein the annealing temperature of the polymerase chain reaction is from 44° C. to 52° C.
